(19) United States
(12) Patent Application Publication
(10) Pub. No.: US 2010/0093794 A1
(43) Pub. Date: Apr. 15, 2010

(54) Use of a Compound in Obtaining Cytoskeleton Blockage and Cell Elongation

(76) Inventors: Jui-I CHAO, Hsinchu City (TW); Tzu-Sheng HSU, Changhua County (TW); Chinpiao CHEN, Hualien City (TW); Pei-Ting LEE, Taipei City (TW); Shu-Jun CHIU, Hsinchu County (TW)

Correspondence Address: Dr. BANGER SHIA;Patent Office of Bang Shia, 102 Lindencrest Ct., Sugar Land, TX, 77479-5201, US

(21) Appl. No.: 12/626,926

(22) Filed: Nov. 29, 2009

Related U.S. Application Data
(63) Continuation-in-part of application No. 11/548,803, filed on Oct. 12, 2006.

(51) Int. Cl. A61K 31/4709; A61P 35/00

(52) U.S. Cl. 514/314

(57) ABSTRACT

A use of a compound in obtaining cytoskeleton and cell elongation is disclosed, the compound is 7-chloro-6-piperidin-1-yl-quinoline-5,8-dione with a chemical formula of C₁₄H₁₃CIN₂O₂, is designated as PT-262. The PT-262 can induce cell elongation by stabilization of the F-actin and induction of the abnormal actin polymerization in cancer cells, further, the PT-262 possesses antitumor activity and can block survival pathway of the cancer cells, resulting in cancer cells apoptosis, and the PT-262 can induce growth arrest and inhibition of cell cycle. PT-262 stabilizes cancer cells cytoskeleton that results in an irreversible cell elongation, decreases the levels of cyclin B1 and phospho-cdc2 proteins, and inhibits the survival signal pathway of Ras-ERK proteins. The PT-262 also inhibits the mitochondrial membrane potential and induces the caspase-3 activation and apoptosis in the cancer cells.

This application is a continuation of part of U.S. patent application Ser. No. 11/548,803, which claims the benefit of the earlier filing date of Oct. 12, 2006. Claim 1 of this application is revised from the previous claim 1 of the U.S. patent application Ser. No. 11/548,803, claim 2 of this application is revised from the previous claim 3 of the U.S. patent application Ser. No. 11/548,803, claim 3 of this application is revised from the previous claim 5 of the U.S. patent application Ser. No. 11/548,803, claim 4 of this application is new, claim 5 of this application is revised from the previous claim 7 of the U.S. patent application Ser. No. 11/548,803, claim 6 of this application is revised from the previous claim 9 of the U.S. patent application Ser. No. 11/548,803, claim 7 of this application is revised from the previous claim 11 of the U.S. patent application Ser. No. 11/548,803.

### BACKGROUND OF THE INVENTION

1. Field of the Invention

The present invention relates to a use of a compound in obtaining cytoskeleton blockage and cell elongation, 7-chloro-6-piperidin-1-yl-quinoline-5,8-dione.

2. Description of the Prior Art

Poor eating habits, improper lifestyle and Physical inactivity are the main risk factors of getting cancer. And the cancer is now increasingly affecting young patients. Cancer is only curable in its early stages, therefore, how to control the illness and prevent deterioration are issues of great importance facing the modern medicine at present.

Of new discoveries of anticancer agents, paclitaxel and Colchicumautumatal are proven to have noticeable tumor-suppressing effect; however, they also have some problems. Please refer to the following drug descriptions.

Paclitaxel:

The research group of NCI (national cancer institute) led by MonroeWall confirms that paclitaxel is effective against several cancers, and particularly effective against breast adenocarcinoma and ovary adenocarcinoma. Of 40 terminal cancer patients treated with paclitaxel, 12 have been reduced in tumor size by over 50%, with a suppression rate of 30%.

Paclitaxel (Taxol) is a compound possessing the taxane C15-ring, which contact with an unsaturated 4 member-O-ring at C4 and C5, and an ester side chain at 13-C. There are 9 optical carbon atoms in paclitaxel. Through the study on structure-activity relationship of the paclitaxel, we found that the ester side chain is the key point for enabling the paclitaxel molecule to have pharmacological Activity, although it decreases the solubility of the paclitaxel molecule in water. Changing the side chain by removing any group at 13-C can increase the solubility of the molecule; however, the activity of the molecule will also be reduced. In addition, further, the paclitaxel did not induce the cell elongation, and can't stabilize the actin filaments and repress the actin polymerization.

In addition, paclitaxel is extracted from the bark of the Pacific yew tree, and the paclitaxel content in the bark of the Pacific yew tree is 0.01%-0.03%. About 9000 kg bark from 2000-3000 yew trees provides only 1 Kg paclitaxel, and treating a patient requires the use of 5-6 100-year old yew trees. The Pacific yew tree is a slow growing tree, producing paclitaxel from the yew tree will cause severe damage to the environment. Therefore, it must find another way to produce the paclitaxel.

Colchicumautumatal:

Colchicumautumatal is a perennial herb and bulb plant of lily family, blooms from August to December, its bud is spindly, and flower is funnel-shaped and bright pink (or purplish red). As early as in 19^{th} century, colchicine has been used to treat sciatica and arthritis, and afterwards, it was found that colchicines has certain therapeutic effect on breast adenocarcinoma, ovary adenocarcinoma, and Acute Lymphocytic Leukemia. Colchicine (antitumor drug) is effective against breast adenocarcinoma, and has certain therapeutic effect on ovary adenocarcinoma, carcinoma of esophagus, lung adenocarcinoma.

However, Colchicumautumatal has serious toxic side effect, and the primary symptom is the gastrointestinal reaction: such as nausea, vomiting, diarrhea, abdominal pain. Further, Colchicumautumatal can inhibit the bone marrow, causing anemia and agranulocytosis. Likewise, Colchicumautumatal did not induce the cell elongation, and can't stabilize the actin filaments and repress the actin polymerization.

The present invention has arisen to mitigate and/or obviate the afore-described disadvantages of the conventional anticancer drugs.

### SUMMARY OF THE INVENTION

The present invention provides a use of a compound in obtaining cytoskeleton and cell elongation, 7-chloro-6-piperidin-1-yl-quinoline-5,8-dione, which is designated as PT-262.

PT-262 possesses antitumor activity, induces the apoptosis of various human cancer cells (including lung cancer, breast cancer, cervical cancer, etc), and induces growth arrest and inhibition of cell cycle. PT-262 stabilizes cancer cells cytoskeleton that results in an irreversible cell elongation, decreases the levels of cyclin B1 and phospho-cdc2 proteins, and inhibits the survival signal pathway of Ras-ERK proteins. The PT-262 also inhibits the mitochondrial membrane potential and induces the caspase-3 activation and apoptosis in the cancer cells.

The 5,8-quinolinedinoes and 6,7-dihaloquinoline-5,8-diones are useful precursors for producing multiple types of bioactive products. The derivatives of quinolinediones have been shown to possess biological activities including antitumor and antimicrobial actions. 6-Anilno-5,8-quinolinedione, an inhibitor of guanylyl cyclase, inhibits cell proliferation and induces cellular senescence in tumor cells. 6-Chloro-7-(2-morpholin-4-ylethylamino) quinoline-5,8-dione, a potent inhibitor of cdc25 protein phosphatases, blocks the proliferation of human breast cancer cells. In addition, lavendamycin is a bacterially derived quinolinedione that displays antitumor activity.

It is important that the PT-262, as a new derivative of 5,8-quinolinedione, can alter the cytoskeleton and noticeably induces the cell elongation.

The cytoskeleton of microtubules and actin filament (F-actin) has been proposed as the potent targets for cancer chemotherapy. For example, paclitaxel (taxol) can stabilize microtubules and induces the formation of microtubule bundles to block the mitosis progression. In contrast, the vinca alkaloids and colchicines induce the mitotic arrest by inhibiting microtubule polymerization and destroying the mitotic spindle. Cytochalasins bind to the plus end of F-actin, reduces F-actin mass, and prevent actin polymerization. However, the phalloidin can bind and stabilize the side of F-actin that plays resulting in the inhibition of actin depolymerization.

PT-262 is similar with phalloidin on the induction of cell elongation by abnormal actin polymerization. The actin dynamics and remodeling are regulated by the activation of actin signaling pathways including the small GTPase proteins such as Ras, Rac, Rho, and cdc42. The regulation of these pathways can affect the stabilization of the cytoskeleton. PT-262 can inhibit the expression of the Ras protein, and can inhibit the pathways of the Ras and other small GTPase proteins, and modulate the polymerization of the F-actin and the cell elongation.

Blockade of survival pathways in tumor cells is an important strategy in cancer therapy. Ras, an oncogenic protein, mediates the extracellular signal regulated-protein kinase (ERK) signal pathway for cell survival and transformation. PT-262 inhibits the Ras-ERK survival signal pathway and provides an antitumor action. The cdc 2 interacts with cyclin B1 that has been shown to play a critical role in the mitotic progression. The activation of the cdc 2 and the cylin B1 is required for mitotic progression. Activation of cdc2 is through the phosphorylation of Thr-161 by cdc2 activating kinase and the dephosphorylation by cdc 25. PT-262 can reduce the level of phospho-cdc2 (Thr-161) and cyclin B1 proteins and block the cell cycle in cancer cells. Furthermore, anticancer drugs can produce antitumor action by inducing the apoptosis pathway in the cancer cells. PT-262 inhibits the mitochondrial membrane potential and induces the caspase-3 activation and apoptosis in the cancer cells.

FIG. 1 shows the chemical structure of the present invention which is expressed as: 7-chloro-6-piperidin-1-yl-quinoline-5,8-dione, and its chemical formal is: C₁₄H₁₃CIN₂O₂.

A process for synthesizing a compound capable of cytoskeleton and induction of cell elongation, comprising the steps of:

Triethylamine of 0.56 mL, 5.1 mmol was added dropwise to the solution of 6,7-dichloroquinoline-5,8-dione of 1.00 g, 4.4 mmol and the piperidine of 0.50 mL, 5.1 mmol in 150 ml of benzene with stirring at room temperature for 5 minutes, and the solvent was removed using evaporator to give a dark brown solid, the PT-262 was purified by flash chromatography using 50% ethyl acetate/hexanes to elute, yielding 0.48 g, 40% of 6-chloro-7-piperidin-1-yl-quinoline-5,8-dione and 0.72, 59% of PT-262.

The configuration of PT-262 was identified by 2D NMR spectra, the molecular weight of the PT-262 IS 276.0666, and the melting point is 145-145° C.

Therefore, the PT-262 stabilizes cancer cells cytoskeleton that results in an irreversible cell elongation, induces growth arrest and apoptosis of cancer cells and inhibition of cell cycle. In addition, PT-262 also alters the structure of the cytoskeleton and the extracellular matrix.

### BRIEF DESCRIPTION OF THE DRAWINGS

FIG. 1 is a chemical structure of the PT-262 in accordance with the present invention;

FIG. 2A is a data diagram of showing effect of PT-262 on the induction of apoptosis in lung carcinoma cells;

FIG. 2B is a data diagram of showing effect of PT-262 on the induction of apoptosis in breast adenocarcinoma cells;

FIG. 2C is a data diagram of showing effect of PT-262 on the induction of apoptosis in cervical carcinoma cells;

FIG. 3 shows the effect of PT-262 on the cell growth in cancer cells;

FIG. 4 shows the effect of PT-262 on the cell cycle progression in cancer cells;

FIG. 5 shows that PT-262 decreases the levels of cyclin B1 and phospho-cdc2 proteins, and the expression levels of Ras and phospho-ERK;

FIGS. 6A-6B show the analysis on the influence of the PT-262 on the Mitochondrial membrane potential of the cancer cells (FIG. 6A shows that as the drug concentration increased, the Mitochondrial membrane potential of the A549 lung carcinoma cells were noticeably inhibited, FIG. 6B shows that PT-262 noticeably induced the caspase-3 activation;

FIGS. 7A-7B show the induction of the actin filaments polymerization and cell elongation by PT-262 in the cancer cells (FIG. 7A shows the micrograph, and FIG. 7B shows the calculation of the cell length under the Leica confocal software);

FIG. 8 shows the comparison of the PT-262 and a variety of cytoskeleton inhibitors.

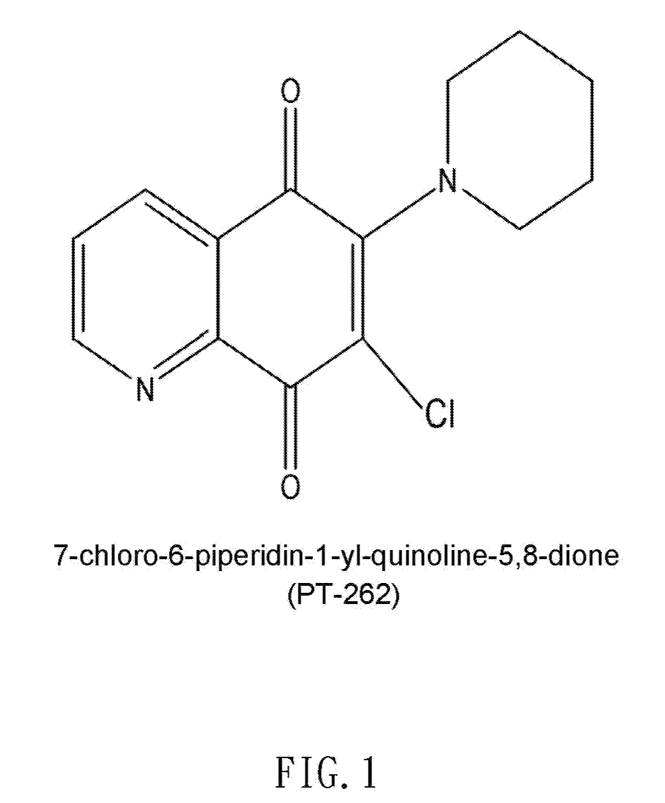

Table 1 shows the comparison of the PT-262 and a variety of cytoskeleton inhibitors.

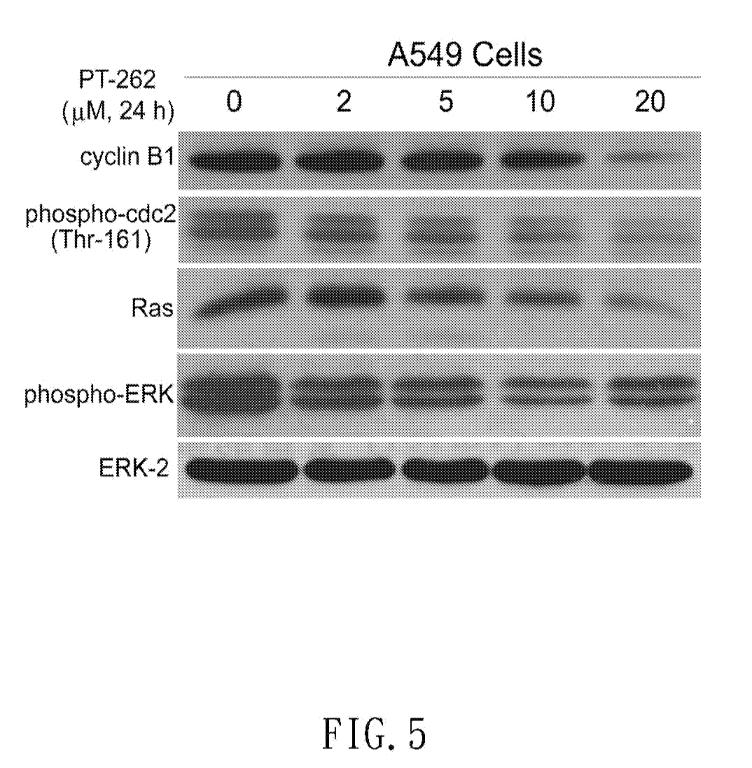

Appendix: the photographs of FIGS. 5, 6, 7 and 8.

### DETAILED DESCRIPTION OF THE PREFERRED EMBODIMENTS

The present invention will be more clear from the following description when viewed together with the accompanying drawings, which show, for purpose of illustrations only, the preferred embodiment in accordance with the present invention.

The cell culture of PT-262, the experimental procedures, and the experiment results are described in conjunction with the accompanying drawings.

Cell Culture

The A549 cell line was derived from lung carcinoma of a 58-year-old male. The H1299 cell line has a homozygous deletion of the P53 gene that was derived from a non-small cell lung adenocarcinoma tumor. MCF-7 cell line was derived from breast adenocarcinoma of a 69-year-old Caucasian female. Hela cell line was derived from cervical carcinoma of a 31-year-old female. These cell lines are cultured in RPMI-1640 medium supplemented with 10% fetal bovine serum, 100 units/ml penicillin, 100 ug/ml streptomycin, and L-glutamine (0.03%, w/v), and cells were incubated at 37° C. and 5% CO₂.

MTT Assay

Briefly, the cells were plated in 96-well plates at a density of 1×10⁴ cells/well for 16-20 hours. Then the cells were treated with pt-262 for 24 hours in serum-free RPMI-1640 medium. After drug treatment, the cells were washed with phosphate-buffered saline (PBS), and were re-cultured in complete RPMI-1640 medium for 48 hours. Subsequently, the medium was replaced and the cells were incubated with 500 ug/ml MTT in complete RPMI-1640 medium for 4 hours. The surviving cells were dissolved in DMSO after removing the MTT medium, and was measured at 565 nm using a ELISA reader.

Cell Growth Assay

The cells were plated at a density of 5×10⁵ cells per p100 Petri dish for 16-20 hours. Then the cells were treated with PT-262 of different concentrations for 24 hours. After drug treatment, the cells were washed with PBS and re-treated with trypsin, the cells were suspended and were counted by a hemocytometer.

Cell Cycle Analysis

The cells were plated at a density of 1×10⁶ cells per p60 dish for 16-20 hours. Then the cells were treated with PT-262 for 24 hours. After drug treatment, the cells were washed with PBS and re-treated with trypsin, the cells were suspended and collected in a 15 ml centrifuge tube. After centrifugation at 1500 rpm for 5 minutes, the cells were fixed with 70% ethanol and stored at −20° C. for at least 2 hours. After being re-centrifuged at 1500 rpm for 5 minutes, the cell pellets were incubated with 4 ug/ml propidium iodine solution containing 1% Triton X-100 and 100 ug/ml RNase A for 30 minutes. The cell cycle was then analyzed by flow cytometer, and the percentage of cell cycle was quantified by ModFit Lt software (Ver. 2.0).

Mitochondrial Membrane Potential

The cells were cultured in 60-mm Petri dish at a density of 5×10⁵ cells. Then the cells were treated with PT-262 of different concentrations for 24 hours. After drug treatment, the cells were washed with PBS and were trypsinized, the cells were suspended and were counted by a hemocytometer. The cells were collected by centrifugation, and the pellets were resuspended in 70% ethanol and stored at ˜20° C. for at least 2 hours. After centrifugation, the pelts were incubated with 0.5 uM DiOC6 for 30 minutes. Then cell pellets were collected by centrifugation and resuspended in 0.5 ml ice-cold PBS. Finally, fluorescence intensities of DiOC6 were analyzed on a flow cytometer.

Western Blot

At the end of treatment, the cells were lysed in the cell extract buffer containing the protease inhibitors. Amounts of proteins in samples were subjected to electrophoresis using 10-12 sodium dodecyl sulfate-polyacrylamide gels. After electrophoretic transfer of proteins onto polyvinylidene difluoride (PVDF) membranes, the membranes were dipped in 5% degreased milk containing first antibody for 24 hours at 4° C. After being washed three times with TTBS buffer solution for 5-15 minutes at room temperature, the PVDF membranes are dipped in 5% degreased milk containing second antibody for 1-2 hours at room temperature. And then the membranes were re-washed three times with TTBS buffer solution for 5-15 minutes at room temperature. Finally, the protein bands were visualized using the enhanced chemiluminescence detection system.

Cytoskeleton Staining and Confocal Microscope

The cells were cultured on coverslips kept in a p60 Petri dish, and the coverslips were kept in a CO₂ incubator for 16-20 hours. After being treated with or without PT-262, the cells were fixed in 4% parafomaldehyde solution for 60 minutes at 37° C. Then the coverslips were washed three times with PBS. The F-actin and β-tubulin were stained with 20 U/ml BODIPY FL phallacidin and anti-β-tubulin Cy3 for 30 minutes at 37° C., respectively. Finally, the nuclei were stained with 2.5 ug/ml Hoechst 33258 for 30 minutes. And the cells were added with 80% Glycerin and sealed with nail varnish. The samples were examined under a Leica confocal laser scanning microscope.

Statistical Analysis

All results were obtained at least from three separate experiments. Data were analyzed using Student's t test, and significant differences between values obtained from the population of cells treated with different conditions were compared. A p value of <0.05 was considered as statistically significant.

Results

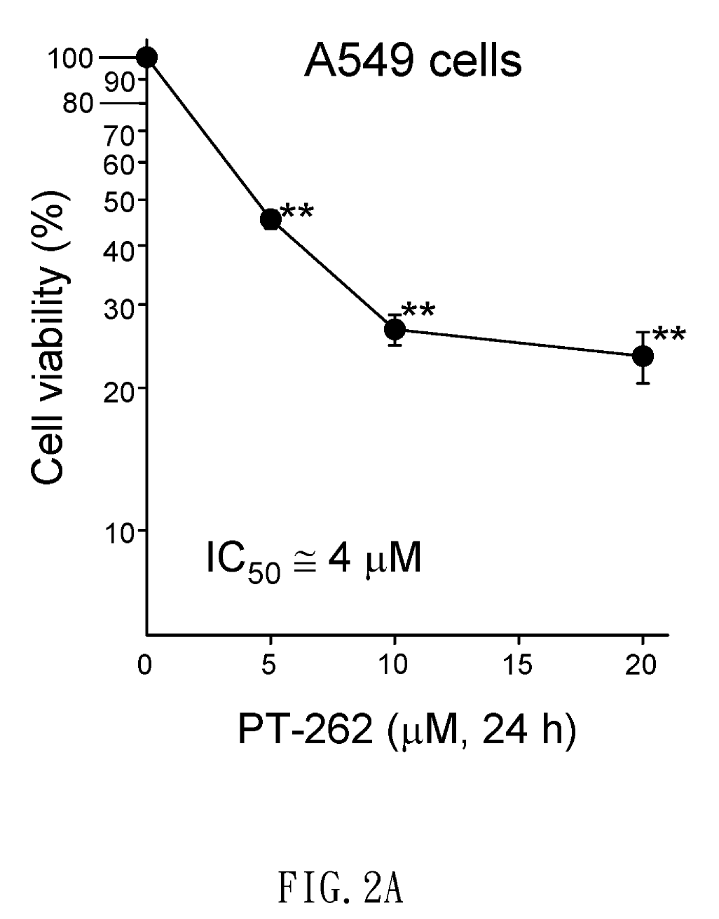

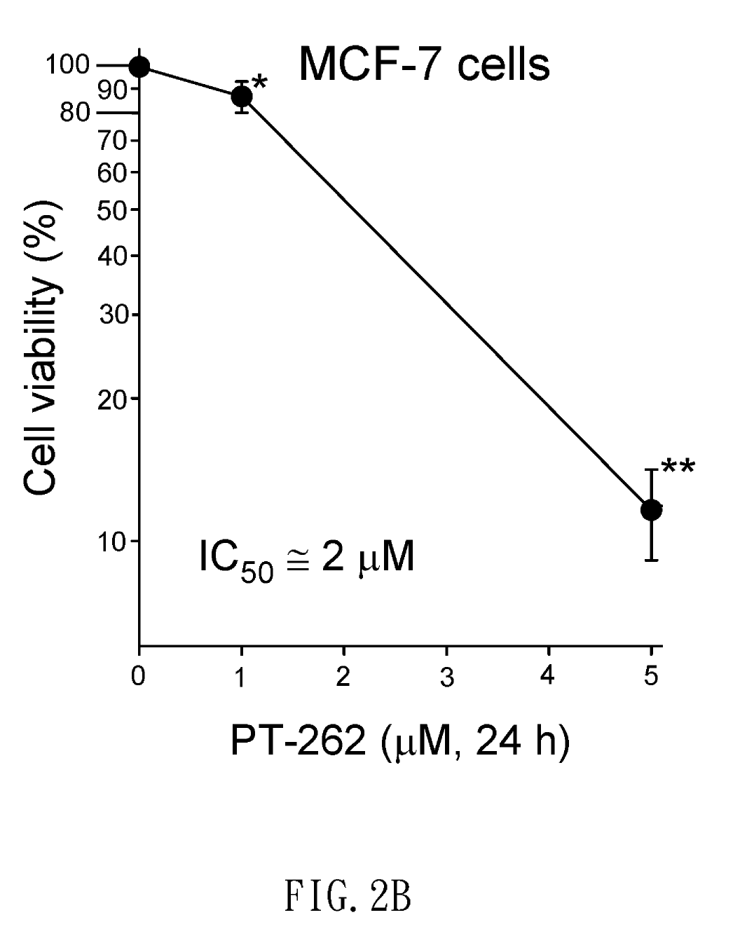

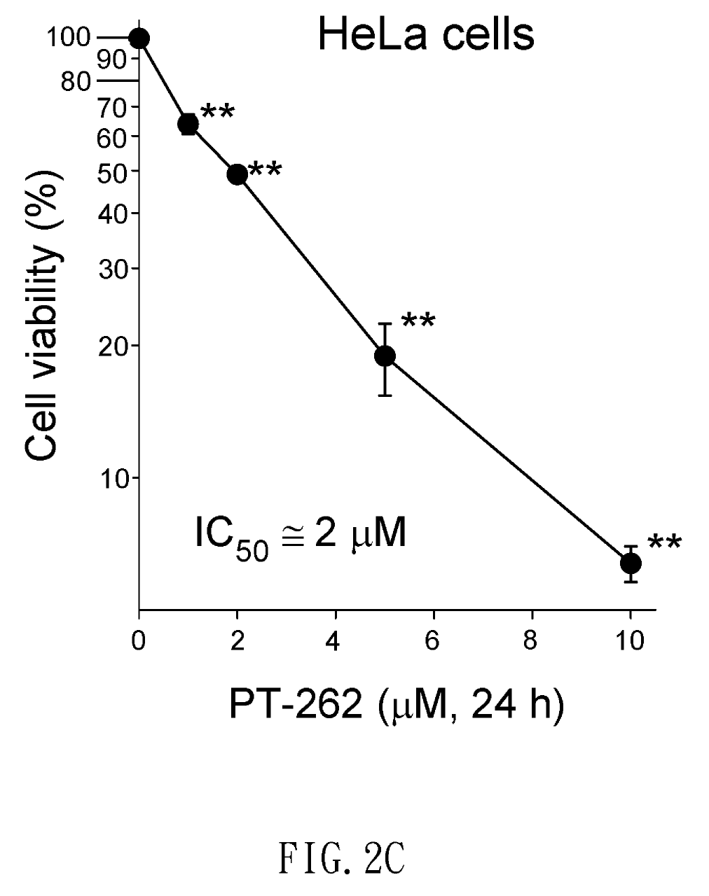

The results of Cytotoxicity were analyzed by the MTT assay and were obtained from 3-14 experiments. * means p<0.05, ** means p<0.01, in comparison with treatments with and without PT-262. Treatment with 1-10 uM PT-262 for 24 hours, the cell survival ratio of the human A549 lung carcinoma cells (FIG. 2A), MCF-7 breast carcinoma cells (FIG. 2B), and Hela cervical carcinoma cells (FIG. 2C) decreased as the concentration increased. The values of IC50 were around 2-4 uM for the above cancer cell lines examined (at cell viability of 50%) (FIGS. 2A-C).

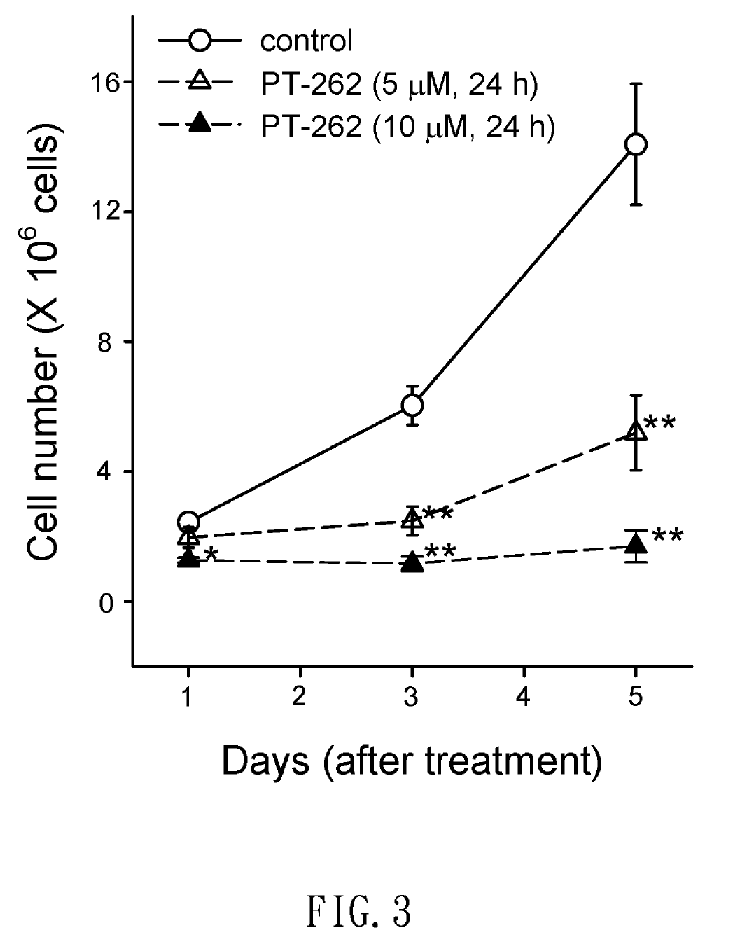

For a better understanding of the present invention, please refer to FIG. 3 again. Analyzed the inhibition of A549 lung carcinoma cells by PT-262, results were obtained from 3 experiments. * means p<0.05, ** means p<0.01, in comparison with treatments with and without PT-262. Treatment with 5-10 uM PT-262 for 24 hours concentration-dependently inhibited the cell growth in A549 lung carcinoma cells (FIG. 3). 10 uM PT-262 for 24 hours treatment almost completely induced the growth arrest (FIG. 3).

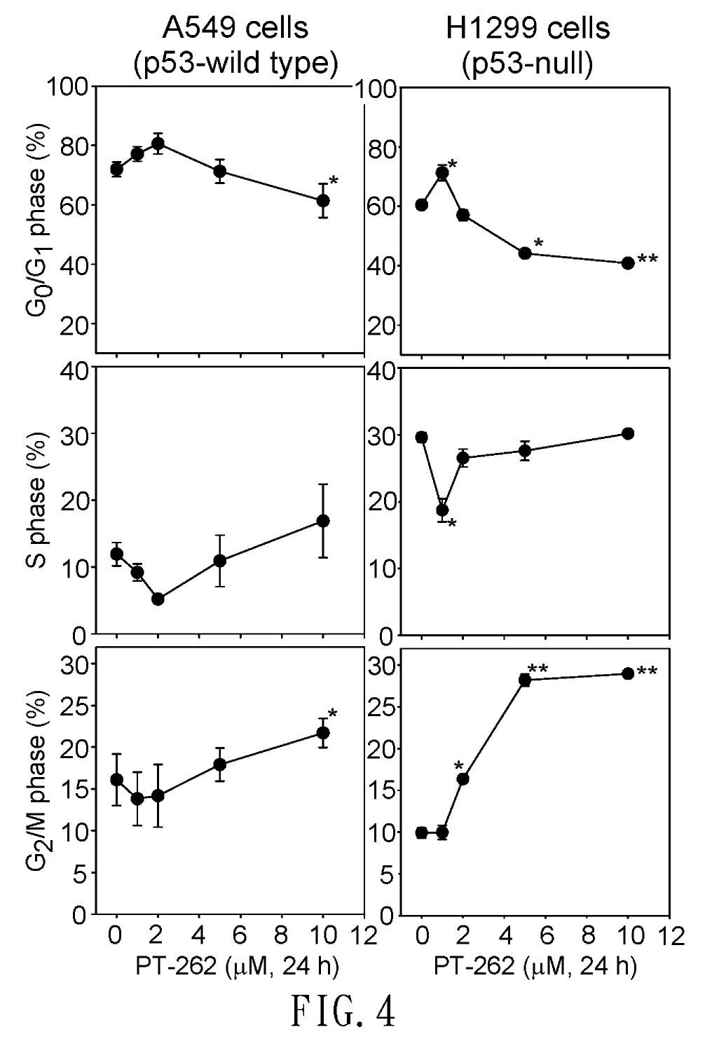

Reference is made to the following descriptions taken in conjunction with FIG. 4, which shows the PT-262 induced the cell grow arrest in the cancer cells. Analyzed the inhibition of A549 lung carcinoma cells (with p53 gene) and H1299 cell lines by PT-262, results were obtained from 3-4 experiments. * means p<0.05, ** means p<0.01, in comparison with treatments with and without PT-262. Treatment with PT-262 decreased the G0/G1 fractions while increased the G2/M fractions in both A549 and H1299 cells (FIG. 4).

FIG. 5 showed that PT-262 inhibited the expression levels of cyclin B1, phospho-cdc2 proteins, Ras and phospho-ERK. The present invention analyzed the influence of PT-262 on the proteins in the cancer cells. FIG. 5 showed the analysis on the expression of the proteins after treatment with PT-262, the analysis shows that PT-262 (5-20 uM, 24 hours) noticeably decreased the levels of cyclin B1 and phospho-cdc2 proteins, and the expression levels of Ras and phospho-ERK also decreased after treatment with PT-262. The activation of ERK is through its phosphorylation, however, ERK-2 was used an internal control, means that the total proteins of ERK are usually not altered.

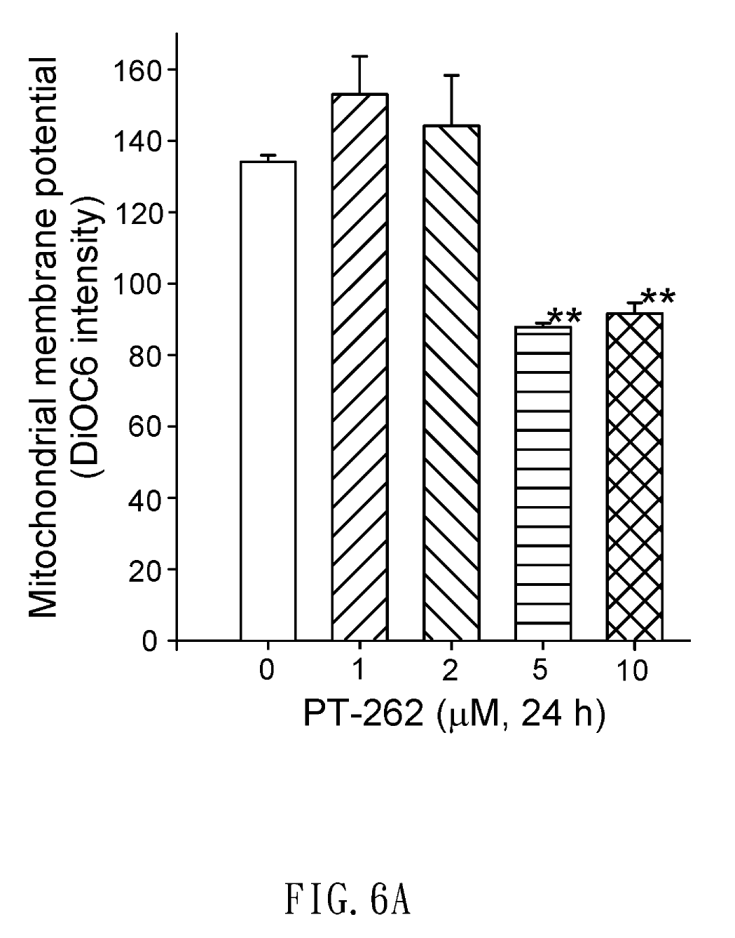

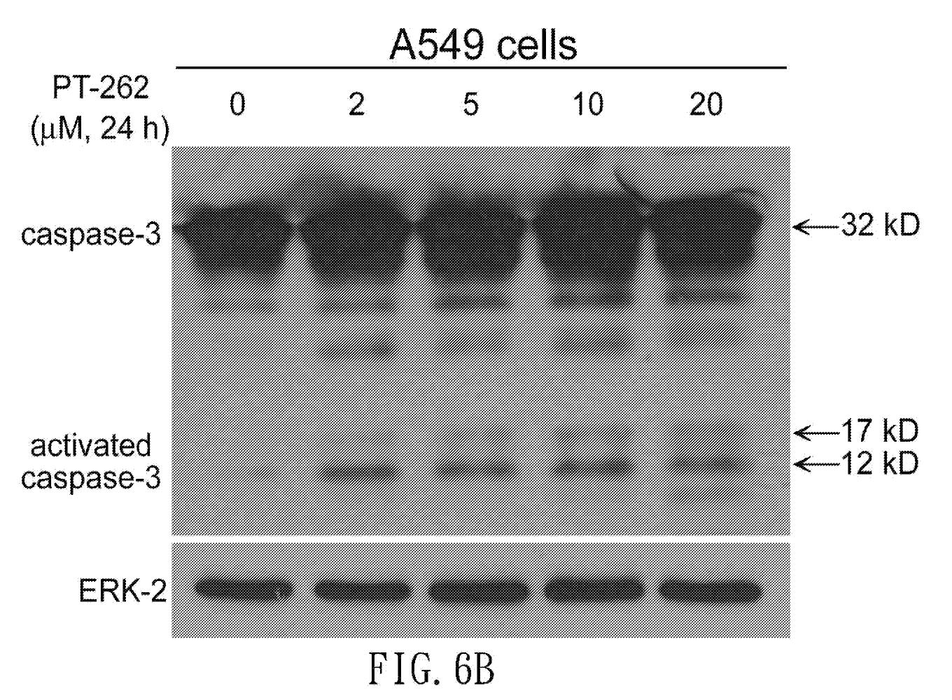

FIG. 6 showed the analysis on the influence of the PT-262 on the Mitochondrial membrane potential of the cancer cells. After treatment with PT-262, the cells were stained with DiOC6, and the cell cycle was then analyzed by flow cytometer. The results were obtained from 3-4 experiments. ** means p<0.01, in comparison with treatments with and without PT-262. As the drug concentration increased, the Mitochondrial membrane potential of the A549 lung carcinoma cells were noticeably inhibited (FIG. 6-A). The influence of the PT-262 on the level of the caspase-3 activation in the cancer cells was analyzed by using the west blot, the analysis showed that PT-262 noticeably induced the caspase-3 activation (FIG. 6B) and apoptosis in the cancer cells.

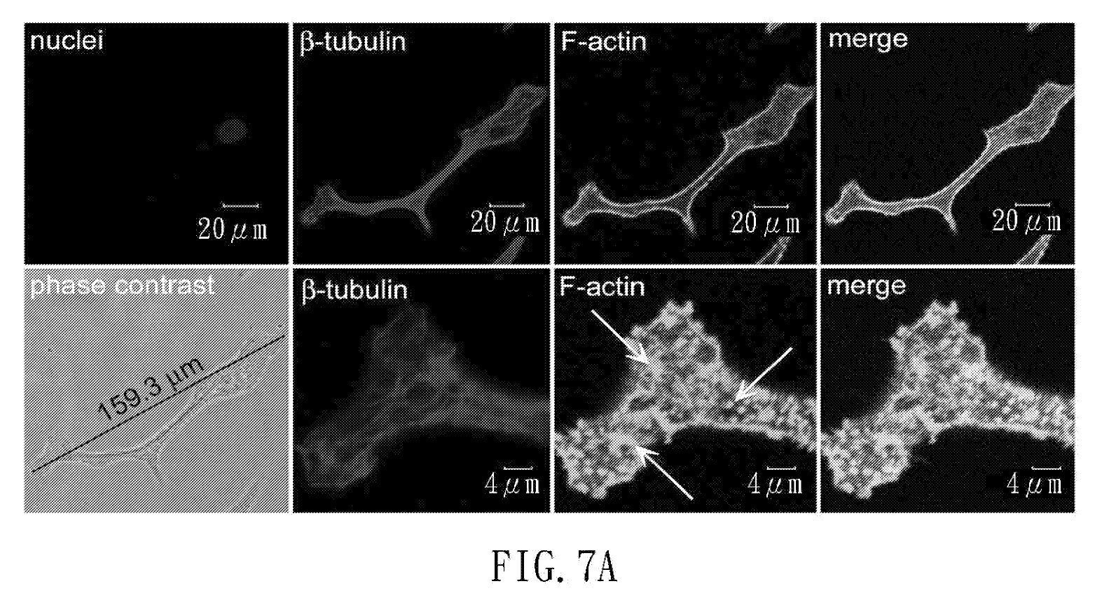

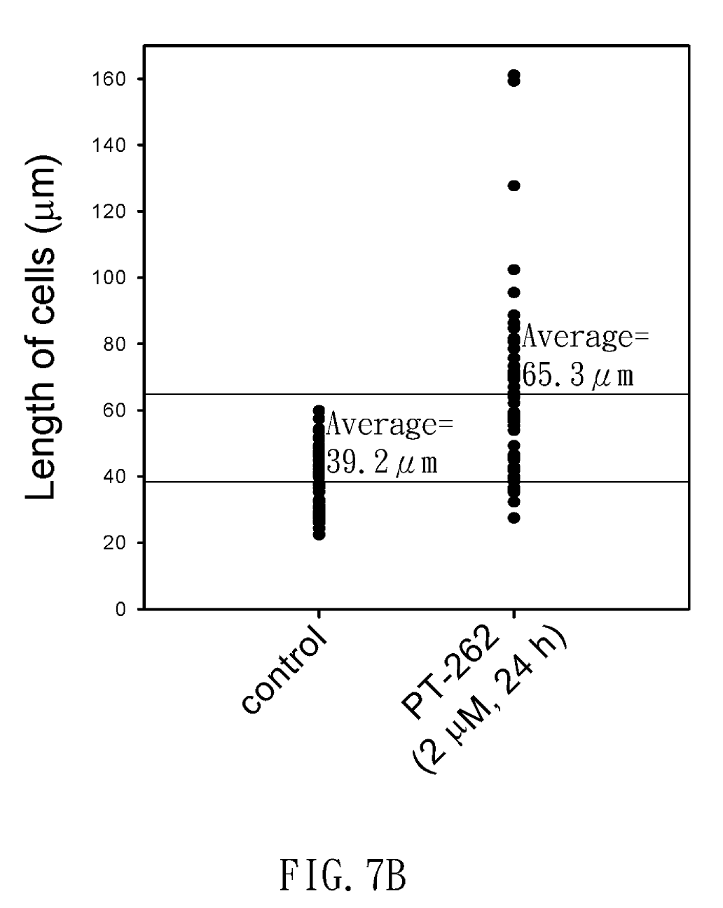

FIG. 7 shows the induction of the actin filaments polymerization and cell elongation by PT-262 in the cancer cells, wherein the β-tubulin, the actin filament, and the nuclei were stained with anti-β-tubulin Cy3, BODIPY FL phallacidin, Hoechst 33258, respectively. And they were examined under a Leica confocal laser scanning microscope. As shown in FIG. 7A, the blue color represented the nuclei of the A549 lung carcinoma cells, the pink color indicated for the β-tubulin, and the green color indicated for the actin filaments. The results showed that PT-262 dramatically induced the alternation of cell morphology and the cell shape became longer following treatment. The arrows indicated the actin filament polymerization and the formation of a spike. Calculation of the cell length under the Leica confocal software, the average cell length was increased from 39.2 to 65.3 um (as shown in FIG. 7B), and some cells were increased to 160 um, and the PT-262 also induced the elongation of other various cancer cells.

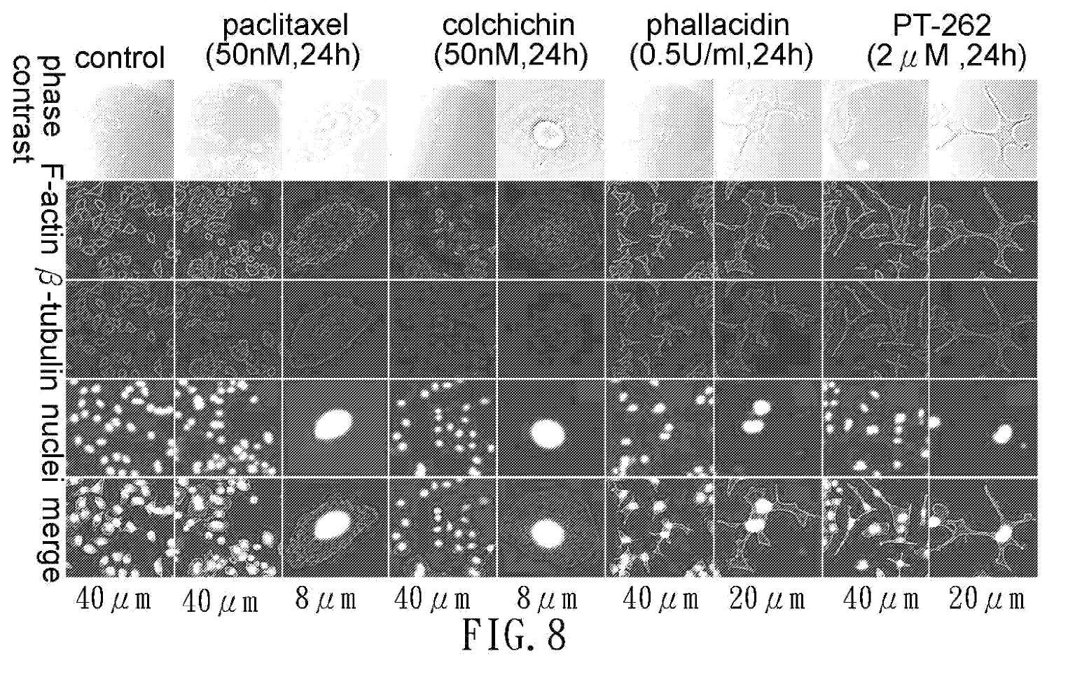

To determine the mechanism of cell elongation induced by PT-262, the A549 cells were compared with a variety of cytoskeleton inhibitors including paclitaxel, colchichine, phalloidin, and cytochalasin B. FIG. 8 showed that 50 nM paclitaxel for 24 hours treatment increased the red fluorescence intensity of β-tubulin from induction of the microtubulin polymerization. In contrast, colchicines (50 nM, 24 hours) reduced the red fluorescence intensity of β-tubulin by inhibition of the microtubulin polymerization. Phallacidin (0.5 U/ml, 24 hours) increased the green fluorescence intensity of F-acin by promotion of the actin polymerization and subsequently caused the cell elongation. Treatment with 2 uM PT-262 also induced the F-actin polymerization and the cell elongation in A549 cells.

In comparison with various actions of cytoskeleton inhibitors (as shown in Table 1), we found that paclitaxel stabilized microtubules and induced microtublin polymerization to block the mitosis progression. Colchicines induced the mitotic arrest by inhibiting microtubule polymerization and destroying the mitotic spindle. Cytochalasins bound to the plus end of F-actin and prevented actin polymerization.

Paclitaxel, colchicines, and cytochalasin didn't induce the cell elongation. However, the phalloidin can bind and stabilize the side of F-actin, and inhibit the actin deploymerization. Like the phalloidin, PT-262 also increased the cell elongation by induction of the actin polymerization in lung carcinoma cells.

Therefore, the compound and the synthesis process of the present invention not only induced the elongation of the cancer cell, but also stabilized the side of F-actin, inhibited the actin deploymerization, affected the structure of the cytoskeleton and the extracellular matrix. Other special functions of the present invention are described as follows:

1. PT-262 can increase the cell elongation by stabilization of the F-actin and induction of the actin polymerization in carcinoma cells.

2. As an inhibitor of the cdc2 activating kinase and the cdc 25, PT-262 can also inhibit the corresponding proteins downstream of the cdc2 and cdc 25.

3. PT-262 inhibited the Ras-ERK survival signal pathway, including all the corresponding upstream and downstream proteins, and prevented the survival, proliferation, and transformation of the cancer cells.

4. PT-262 increased the cell elongation, decreased the mitochondrial membrane potential, and induced the caspase-3 activation and its upstream and downstream proteins.

5. PT-262 stabilized the side of F-actin, inhibited the actin deploymerization of the cancer cells, and affected the structure of the cytoskeleton and the extracellular matrix.

6. PT-262 can stabilize the side of F-actin, inhibit the actin deploymerization of the other cells.

In addition, other derivatives of 7-chloro-6-piperidin-1-yl-quinoline-5,8-dione are also within the scope of the present invention and possess the aforementioned functions and effects.

While we have shown and described various embodiments in accordance with the present invention, it is clear to those skilled in the art that further embodiments may be made without departing from the scope of the present invention.

TABLE 1
various cytoskeleton inhibitors

| | mechanism | | | |
|---|---|---|---|---|
| | Microtubule | Microtubule | Actin | Actin |
| | poly- | depoly- | poly- | depoly- |
| drug | merization | merization | merization | merization |
| paclitaxel | +^{a} | - | - | - |
| colchichine | - | +^{b} | - | - |
| cytochalasin B | - | - | - | +^{c} |
| phalloidin | - | - | +^{d} | - |
| PT-262 | - | - | +^{f} | - |

Inhibit mitotic spindle, induce mitotic arrest, but can't induce cell elongation in cancer cells
Inhibit cytokinesis by induction of F-actin depolymerization, but can't induce cell elongation in cancer cells
Induce cell elongation by induction of the abnormal actin polymerization in cancer cells

What is claimed is:

1. A use of a compound in obtaining cytoskeleton blockage and cell elongation, the compound being 7-chloro-6-piperidin-1-yl-quinoline-5,8-dione.

2. The use of a compound in obtaining cytoskeleton blockage and cell elongation as claimed in claim 1, wherein the compound inhibits cytoskeleton and induces cell elongation.

3. The use of a compound in obtaining cytoskeleton blockage and cell elongation as claimed in claim 1, wherein the compound inhibits cdc2 kinase and cdc 25.

4. The use of a compound in obtaining cytoskeleton blockage and cell elongation as claimed in claim 1, wherein the compound inhibits the cytoskeleton of cancer cells and induces the elongation of the cancer cells, and the cancer cells include cells of lung cancer, breast adenocarcinoma, and cervical carcinoma.

5. The use of a compound in obtaining cytoskeleton blockage and cell elongation as claimed in claim 4, wherein the compound inhibits Ras-ERK survival signal pathway.

6. The use of a compound in obtaining cytoskeleton blockage and cell elongation as claimed in claim 4, wherein the compound induces apoptosis of the cancer cells, decreases mitochondrial membrane potential, and induces caspase-3 activation.

7. The use of a compound in obtaining cytoskeleton blockage and cell elongation as claimed in claim 4, wherein the compound stabilizes the side of F-actin, and inhibits actin deploymerization of the cancer cells.
